(19) United States
(12) Patent Application Publication
(10) Pub. No.: US 2024/0111046 A1
(43) Pub. Date: Apr. 4, 2024

(54) METHOD AND SYSTEM FOR FLOW PROCESSING ON CHANNEL DATA FOR APPLICATION OF NONLINEAR BEAMFORMING

(71) Applicant: GE Precision Healthcare LLC, Wauwatosa, WI (US)

(72) Inventors: Anders R. Sornes, Oslo (NO); Thor Andreas Tangen, Oslo (NO); Bastien E. Denarie, Asker (NO); Kjell Kristoffersen, Oslo (NO)

(21) Appl. No.: 16/681,026

(22) Filed: Nov. 12, 2019

(51) Int. Cl. G01S 15/89; A61B 8/14; G01S 7/52

(52) U.S. Cl. CPC G01S 15/8981; A61B 8/14; G01S 7/52026; G01S 7/5209; G01S 7/52095; G01S 15/8984

(57) ABSTRACT

Systems and methods for enhancing spatial specificity and increasing effective image acquisition speed by performing flow processing on channel data for application of nonlinear beamforming are provided. The method includes generating clutter filtered signals, delaying the clutter filtered signals to provide delay aligned clutter filtered signals, calculating coherency of the delay aligned clutter filtered signals, and nonlinearly combining the delay aligned clutter filtered signals and the coherency of the delay aligned clutter filtered signals across each transducer element at one or more depths to generate at least one beamformed signal for each received set of echo signals in a sequence of echo signals at the one or more depths. The method includes calculating and presenting a measurement for the one or more depths based on the at least one beamformed signal for each received set of echo signals in the sequence of echo signals at the one or more depths.

### FIELD

Certain embodiments relate to ultrasound imaging. More specifically, certain embodiments enhance spatial specificity and increase an effective image acquisition speed by performing flow processing on channel data for application of nonlinear beamforming.

### BACKGROUND

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

Doppler ultrasound imaging uses reflected sound waves to visualize blood flow through a blood vessel. Doppler ultrasound may assist doctors with evaluating blood flow through major arteries and veins, such as those of the arms, legs, and neck. Doppler ultrasound images may show blocked or reduced flow of blood through narrow areas in the major arteries of the neck that could cause a stroke and can reveal blood clots in leg veins (deep vein thrombosis) that could break loose and block blood flow to the lungs (pulmonary embolism). During pregnancy, Doppler ultrasound may be used to look at blood flow in an unborn baby to check the health of the fetus.

Various types of Doppler imaging may be used to analyze flow, such as color flow (CF) imaging, three-dimensional color flow (3DCF) imaging, blood speckle imaging (BSI), and like. Multi-line acquisition (MLA) ultrasound setups may be used to increase frame rates in CF, BSI, and 3DCF ultrasound imaging. In MLA, several beams are received in response to the transmission of a single transmit beam. Consequently, the final image is composed of a plurality of individual receive-transmit beam sets of varying transmit to receive beam spacing. In some systems, broader transmit beams covering a wider sector or volume may be used to provide even higher framerates or volume rates. The broader transmit beams may be achieved by reducing the aperture or defocusing the beam into a plane wave or a diverging wave geometry. As multiple receive lines of significant distance to the transmit beam axis can be deployed, a resolved image of substantial spatial extension may be procured. The drawback of this defocusing technique is that the spatial specificity produced by two-way beamforming is significantly reduced, which leads to an increased level of sidelobes in the vicinity of strong reflectors. In CF, strong reflectors that are moving, such as the base of the valve apparatus, the valves, and moving pericardium located outside the image area, may give rise to false flow signal artifacts inside the image area.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for enhancing spatial specificity and increasing effective image acquisition speed by performing flow processing on channel data for application of nonlinear beamforming, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to perform flow processing on channel data for application of nonlinear beamforming, in accordance with various embodiments.

FIG. 2 is a flow chart illustrating exemplary steps for performing flow processing on channel data for application of nonlinear beamforming, in accordance with various embodiments.

### DETAILED DESCRIPTION

Certain embodiments may be found in a method and system for performing flow processing on channel data for application of nonlinear beamforming. Various embodiments have the technical effect of increasing spatial specificity that is potentially lost with defocused transmit strategies such as diverging or planar beams. Aspects of the present disclosure have the technical effect of avoiding artifacts from strong off-axis moving tissue structures. Certain embodiments have the technical effect of removing MLA artifacts. Various embodiments have the technical effect of increasing the effective image acquisition speed/performance.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, BSI mode, 3DCF mode, PW Doppler, MGD, and/or sub-modes of B-mode and/or CF such as Volume Compound Imaging (VCI), Shear Wave Elasticity Imaging (SWEI), TVI, Angio, B-flow, BMI, BMI Angio, and in some cases also MM, CM, TVD, CW where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

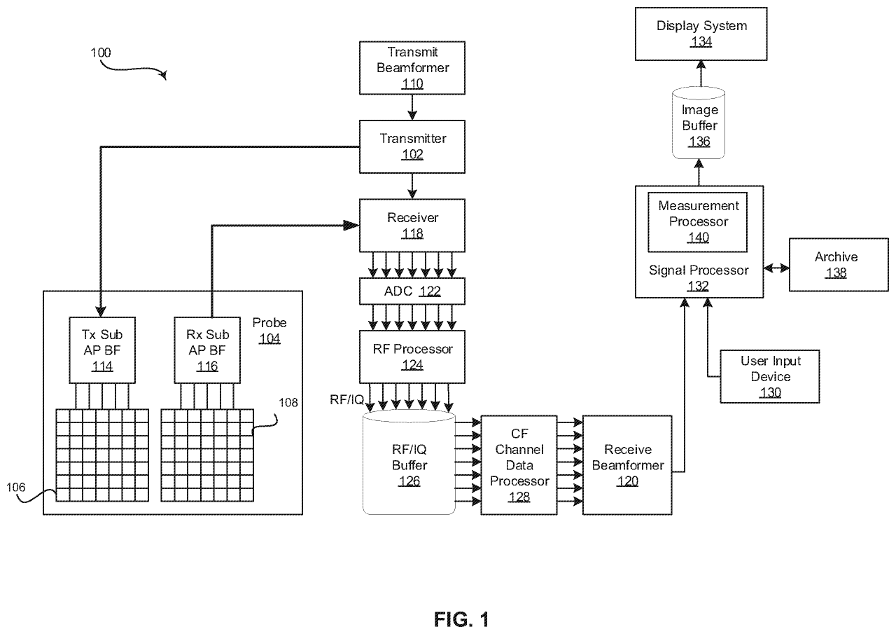

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to perform flow processing on channel data for application of nonlinear beamforming, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, a RF processor 124, a RF/IQ buffer 126, a color flow (CF) channel data processor 128, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements or may be a mechanical one dimensional (1D) array, among other things. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as a heart, a fetus, or any suitable anatomical structure.

The transmit beamformer 110 may comprise suitable logic, circuitry, interfaces and/or code that may be operable to control the transmitter 102 which, optionally through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, optionally undergo sub-aperture beamforming by a receive sub-aperture beamformer 116, and/or are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The color flow (CF) channel data processor 128 may comprise suitable logic, circuitry, interfaces and/or code that may be operable to perform part of or all of the color flow packet processing on channel data, before beamforming is applied. For example, the CF channel data processor 128 may comprise suitable logic, circuitry, interfaces and/or code that may be operable to perform clutter filtering on channel data to attenuate stationary and/or slow reflectors in the channel data. The CF channel data processor 128 receives a sequence of color flow echo signals responsive to a sequence of transmit beams transmitted in a same direction with a fixed pulse repetition frequency. Other transmit beams in other directions may be interleaved or provided sequentially. The incoming echo signal channel data is multiplied with appropriate clutter filter coefficients and summed into buffers containing sub-results from previous partial accumulations to form a full set of clutter filter signals once the packet is completed for a particular transmit beam direction. The CF channel data processor 128 may apply one or more clutter filters, such as FIR filters, eigenfilters, polynomial regression filters, and/or any suitable clutter filter.

In a representative embodiment, the CF channel data processor 128 may provide the clutter filtered signals to the receive beamformer 120. Alternatively, prior to providing the clutter filtered signals to the beamformer 120, the CF channel data processor 128 may perform correlation estimates and/or velocity/bandwidth/power estimates on the clutter filtered signals with the result being augmented with phase information from the original data set. For example, the correlation function estimates R1 and R0 (average lag 1 and lag 0 correlation of the packet) may be processed by the CF channel data processor 128 on channel data and passed on to the beamformer 120. As another example, derived quantities from the correlation functions, such as velocity, bandwidth and Doppler power, may be processed by the CF channel data processor 128 from channel data and passed into the beamformer 120. In both examples, phase information that is vital for localizing the scatterer is removed. Accordingly, the phase of the original IQ data is applied to the derived quantities in order to retain the spatial discrimination to be achieved in the beamforming operation, as this information is lost in the correlation calculation. The phase of the original IQ data may be applied to the correlation estimates by the CF channel data processor 128 by applying the phase of one of original IQ data samples. Alternatively, the phase of the original IQ data may be applied by the CF channel data processor 128 by applying an average over several of the incoming IQ data samples and these may be corrected for the phase rotation anticipated from the calculated average velocity or lag 1 correlation between successive clutter filtered samples in the packet. The process of performing correlation estimates and/or velocity/bandwidth/power estimates on the clutter filtered signals may further enhance in the beamformer the signal having the average velocity and attenuate the presence of signal components of other motions. The processing in the representative or in these alternative embodiments further eliminates the corner-turning stage, which is a time consuming re-ordering of data in memory to align the echo signals in a packet for subsequent packet processing operations.

The receive beamformer 120 may comprise suitable logic, circuitry, interfaces and/or code that may be operable to perform nonlinear beamforming processing in addition to, or instead of, standard delay and sum of the channel signals received from CF channel data processor 128 to output beam summed receive lines. In various embodiments, the receive beamformer 120 applies nonlinear beamforming techniques that emphasize points in space with high coherency of the delay corrected channel signals. The receive beamformer 120 may be configured to replace, mix, or multiply in a measure of phase coherence into the beam sum in order to weigh down off-axis scattering signals and sidelobe energy. The nonlinear beamforming techniques provided by the receive beamformer 120 are configured to regain spatial specificity for color flow signals. In various embodiments, the clutter filtered signals output by the CF channel processor 128 may be beamformed into a plurality of receive directions or multi-line acquisitions (MLAs) for a single transmit direction. The receive beamformer 120 may comprise suitable logic, circuitry, interfaces and/or code that may be operable to weight the delay-and-sum beamforming with a selection of coherence factors prior to IQ data summation in order to emphasize energy from reflectors in the main beam direction, and attenuate sidelobe energy from off axis scatterers. Alternative non-linear processing could also be implemented, such as minimum variance beamforming that could be combined with the output from the linear beamforming to add spatial specificity.

In certain embodiments, by performing clutter filtering prior to beamforming, nonlinear beamforming techniques may be applied that emphasize phase information and coherency of the moving parts of the signal in order to emphasize scatterers on the receive axis and attenuate off-axis scattering information. In this way, spatial specificity may be regained and Doppler signals from outside the imaging beam arising from sidelobes of strong tissue scatterers may be removed. For example, the Doppler signals from outside the imaging beam may first be attenuated by the clutter filtering process performed by the CF channel data processor 128 and subsequent remaining parts may be attenuated due to lack of coherency by the receive beamformer 120 applying nonlinear beamforming. The processing scheme of providing clutter filtering (and optionally correlation processing) before nonlinear beamforming increases efficiency when using MLA because the clutter filtering (and optionally correlation processing) is applied on the echo signal channel data before the beamformer 120 generates numerous MLA directions in each of which the flow processing is typically applied.

The receive beamformer 120 may apply various techniques for performing nonlinear beamforming. For example, the receive beamformer 120 may apply a coherence factor C that measures coherence as the ratio between coherent and non-coherent summation of the delay aligned channel data, as set forth below:

$$C = \vert {\sum }_{i = 1}^{N} {x}_{i} \vert / {\sum }_{i = 1}^{N} \vert {x}_{i} \vert$$

where x is the delay aligned channel data, i is the channel number, and N is the number of channels in the beamformer. The coherence factor C is multiplied by the receive beamformer 120 in the beamformer output as a factor where tunable adjustment factors can decide to weigh the coherency in with the regular beamformer output to a large or small extent. For purposes of the present disclosure, the terms “coherence” or “coherency” are not limited to the factor C, but include any suitable methods of calculating quantities that are substantially dependent on coherence, see for example, J. Camacho et al., “Adaptive Beamforming by Phase Coherence Processing,” Ultrasound Imaging, Mr. Masayuki Tanabe (Ed.), ISBN: 978-953-307-239-5, InTech, 2011, which is incorporated herein by reference in its entirety. In various embodiments, the coherency factor beamforming may be mixed with regular beamforming. The use of coherency in phase is provided to discriminate and attenuate off axis scatterers and side lobe energy from real in-beam reflectors.

In various embodiments, the resulting processed information may be the beam summed receive lines that are output from the receive beamformer(s) 120 and communicated to the signal processor 132. In accordance with some embodiments, the receiver 118, the plurality of A/D converters 122, the RF processor 124, and the beamformer 120 may be integrated into single beamformer(s), which may be digital. In certain embodiments, the receive beamformer(s) 120 may be multiline ultrasound beamformer(s) configured to produce multiple receive lines in response to each single transmitted beam. The multiline receive beamformer(s) 120 may apply different delays and combine the clutter filtered signals to produce steered and focused lines. In certain embodiments, the above-mentioned nonlinear beamforming techniques may be combined with other reconstruction type methods of reducing side lobe energy such as synthetic transmit beam formation or retrospective synthetic focusing techniques utilizing overlaps between two or more adjacent transmit beams. For example, the receive beamformer(s) 120 may be configured to apply Retrospective Transmit Beamforming (RTB) to provide dynamic transmit focusing and align the transmit lines with corresponding receive lines using time delays computed from a probe geometry to correct the acquired ultrasound data.

Referring again to FIG. 1, the user input device 130 may be utilized to input patient data, scan parameters, settings, select protocols and/or templates, select an imaging mode, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the CF channel data processor 128, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform compounding, such as volume compound imaging (VCI), elevation compound imaging (ECI), or the like. In various embodiments, the signal processor 132 may be operable to perform speckle tracking. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information. In the representative embodiment, the signal processor 132 may comprise a measurement processor 140.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores medical image data, channel data processing instructions, nonlinear beamforming instructions, and measurement instructions, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a measurement processor 140 that comprises suitable logic, circuitry, interfaces and/or code that may be operable to calculate one or more measurements based on the beamformed signals received from the receive beamformer 120. The one or more measurements may include a velocity measurement, power measurement, variance measurement, bandwidth measurement, and/or displacement measurement. For example, the measurement processor 140 may be operable to calculate a displacement field from speckle tracking the beamformed data spatially between successive echoes. The measurement data may be presented at the display system 134 and/or stored at archive 138 or any suitable data storage medium. For example, the measurement processor 140 may present the measurement data as a color flow image superimposed onto a B mode image.

The display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as volume compound images, and/or any suitable information. In various embodiments, the display system 134 is operable to present color flow images corresponding with one or more of velocity measurements, power measurements, variance measurements, and/or bandwidth measurements overlaid on a B mode image. In certain embodiments, the display system 134 is operable to present flow trajectories calculated from performing speckle tracking on the beamformed ultrasound images.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

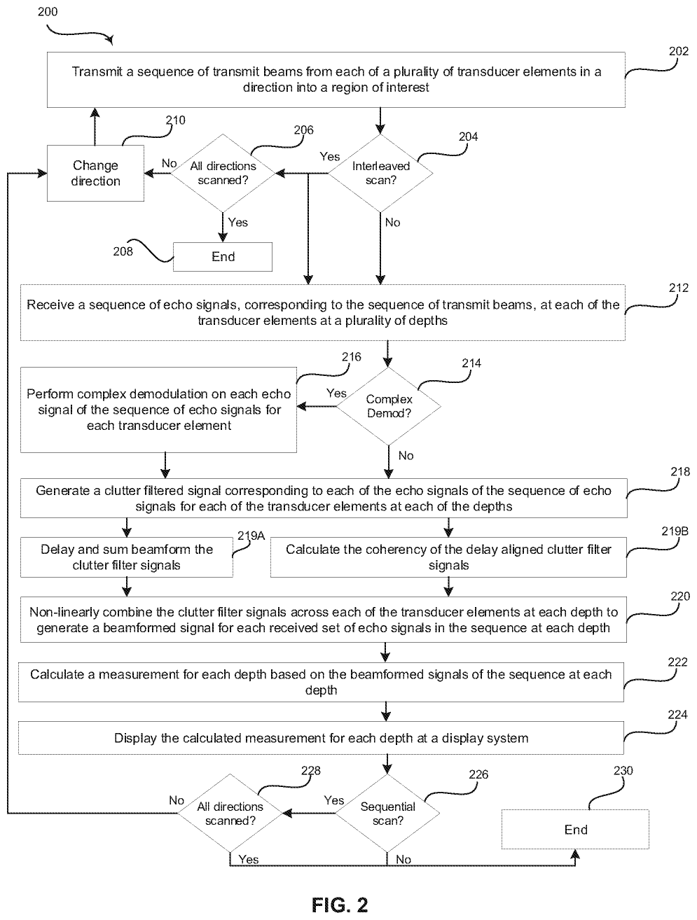

FIG. 2 is a flow chart 200 illustrating exemplary steps 202-230 for performing flow processing on channel data for application of nonlinear beamforming, in accordance with various embodiments. Referring to FIG. 2, there is shown a flow chart 200 comprising exemplary steps 202 through 230. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 202, a sequence of transmit beams are transmitted from each of a plurality of transducer elements 106 in a direction into a region of interest. For example, an ultrasound probe 104 having a group of transmit transducer elements 106 is positioned to acquire ultrasound data in a region of interest. The ultrasound probe transmits a sequence of transmit beams in a direction from each of the transducer elements 106. As an example, each transducer element 106 may transmit ten (10), or any suitable number, of transmit beams sequentially.

At step 204, if the ultrasound scan is an interleaved scan, the process 200 proceeds simultaneously to steps 206 and 212. If the ultrasound scan is a sequential scan, the process 200 proceeds to step 212.

At step 206, if the ultrasound scan is an interleaved scan, the ultrasound system 100 determines whether all directions have been scanned. If all directions have been scanned, the ultrasound probe 104 ceases transmitting additional sequences of transmit beams at step 208. If all directions have not been scanned, the process 200 proceeds to step 210. At step 210, the scan direction is changed and the process 200 returns to step 202 to transmit a sequence of transmit beams from each of the plurality of transducer elements in the new direction into the region of interest.

At step 212, a sequence of echo signals corresponding to the sequence of transmit beams is received at each of the transducer elements 108 at a plurality of depths. For example, an ultrasound probe 104 having a group of receive transducer elements 108, which normally constitute the same elements as the group of transmit transducer elements 106, receive the sequence of echo signals from the region of interest. In various embodiments, the sequence of echo signals comprises a plurality of echo signals corresponding to each transmit beam in the sequence of transmit beams in a multi-line acquisition scan.

At step 214, the process 200 proceeds to step 216 if complex demodulation is to be performed or otherwise proceeds to step 218 if complex demodulation is not being performed. At step 216, complex demodulation is performed on each echo signal of the sequence of echo signals for each transducer element 108. For example, an RF processor 124 may comprise a complex demodulator that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

At step 218, the ultrasound system 100 generates a clutter filtered signal corresponding to each of the echo signals of the sequence of echo signals for each of the transducer elements 108 at each of the depths. For example, a CF channel data processor 128 of the ultrasound system 100 may be operable to perform clutter filtering on channel data to attenuate stationary and/or slow reflectors in the channel data. The CF channel data processor 128 receives a sequence of color flow echo signals responsive to a sequence of transmit beams transmitted in a same direction with a fixed pulse repetition frequency. The incoming echo signal channel data is multiplied with appropriate clutter filter coefficients and summed into buffers containing sub-results from previous partial accumulations to form a full set of clutter filter signals once the packet is completed for a particular transmit beam direction. The CF channel data processor 128 may apply one or more clutter filters, such as FIR filters, eigenfilters, polynomial regression filters, and/or any suitable clutter filter. In various embodiments, prior to proceeding to step 220, the CF channel data processor 128 may perform correlation estimates and/or velocity/bandwidth/power estimates on the clutter filtered signals with the result being augmented with phase information from the original data set.

At step 219A, the ultrasound system 100 may delay and sum beamform the clutter filter signals. For example, a receive beamformer 120 of the ultrasound system 100 may perform standard beamforming on the clutter filter signals. At step 219B, the ultrasound system 100 may calculate the coherency of the delay aligned clutter filter signals.

At step 220, the ultrasound system 100 non-linearly combines the clutter filter signals across each of the transducer elements at each depth to generate at least one beamformed signal for each received set of echo signals in the sequence at each depth. For example, the receive beamformer 120 of the ultrasound system 100 may be operable to combine the standard beamformed clutter filter signals and the calculated coherency of the delay aligned clutter filter signals. In this way, the receive beamformer 120 applies nonlinear beamforming techniques that emphasize points in space with high coherency of the delay corrected channel signals. The nonlinear beamforming techniques may be a coherency factor beamforming technique or any suitable nonlinear beamforming technique that uses coherency in phase to discriminate and attenuate off axis scatterers and side lobe energy from real in-beam reflectors. The nonlinear beamforming techniques provided by the receive beamformer 120 are configured to regain spatial specificity for color flow signals. In various embodiments, the clutter filtered signals output by the CF channel processor 128 may be beamformed into a plurality of receive directions or multi-line acquisitions (MLAs) for a single transmit direction.

At step 222, a signal processor 132 of the ultrasound system 100 calculates a measurement for each depth based on the beamformed signals of the sequence at each depth. For example, a measurement processor 140 of the signal processor 132 may be operable to calculate one or more measurements based on the beamformed signals received from the receive beamformer 120. The one or more measurements may include a velocity measurement, power measurement, variance measurement, and/or bandwidth measurement. In certain embodiments, a speckle tracked displacement measurement may be estimated by comparing beamformed signals from a plurality of depths over the plurality of repeated receive echoes.

At step 224, the signal processor 132 of the ultrasound system 100 may display the calculated measurement for each depth at a display system 134. For example, the signal processor 132 may present the measurement data as a color flow image superimposed onto a B mode image at the display system 134. In various embodiments, velocity trajectories may be presented as a dynamic overlay over the B mode image.

At step 226, if the ultrasound scan is a sequential scan, the process 200 proceeds to step 228. If the ultrasound scan is an interleaved scan, the process 200 ends at step 230.

At step 228, if the ultrasound scan is a sequential scan, the ultrasound system 100 determines whether all directions have been scanned. If all directions have been scanned, the process 200 ends at step 230. If all directions have not been scanned, the process 200 proceeds to step 210. At step 210, the scan direction is changed and the process 200 returns to step 202 to transmit a sequence of transmit beams from each of the plurality of transducer elements in the new direction into the region of interest. The process 200 continues until all directions have been scanned, either sequentially or as an interleaved scan, and all of the ultrasound data is processed and displayed with the measurement data.

Aspects of the present disclosure provide a method 200 and system 100 for enhancing spatial specificity and increasing effective image acquisition speed by performing flow processing on channel data for application of nonlinear beamforming. In accordance with various embodiments, the method 200 may comprise transmitting 202, from each of a plurality of transducer elements 106, 108, a sequence of transmit beams in a direction into a region of interest. The method 200 may comprise receiving 212, at each of the plurality of transducer elements 106, 108 at a plurality of depths, a sequence of echo signals corresponding to the sequence of transmit beams. The method 200 may comprise generating 218, by at least one processor 128, a clutter filtered signal corresponding to each echo signal of the sequence of echo signals for each of the plurality of transducer elements 106, 108 at each of the plurality of depths. The method 200 may comprise delaying 219A, by at least one beamformer 120, the clutter filtered signals to provide delay aligned clutter filtered signals. The method 200 may comprise calculating 219B, by the at least one beamformer 120, a coherency of the delay aligned clutter filtered signals. The method 200 may comprise nonlinearly combining 220, by the at least one beamformer 120, the delay aligned clutter filtered signals and the coherency of the delay aligned clutter filtered signals across each of the plurality of transducer elements 106, 108 at one or more depths to generate at least one beamformed signal for each received set of echo signals in the sequence of echo signals at the one or more depths. The method 200 may comprise calculating 222, by the at least one processor 132, 140, a measurement for the one or more depths based on the at least one beamformed signal for each received set of echo signals in the sequence of echo signals at the one or more depths. The method 200 may comprise presenting 224, by the at least one processor 132, 140, the measurement for the one or more depths at a display system 134.

In an exemplary embodiment, the method 200 may comprise performing 216, by the at least one processor 124, complex demodulation on each echo signal of the sequence of echo signals for each of the plurality of transducer elements 106, 108. In a representative embodiment, the transmitting the sequence of transmit beams is performed in a plurality of directions into a region of interest. In various embodiments, the transmitting the sequence of transmit beams is performed sequentially in a plurality of directions into the region of interest. In certain embodiments, the transmitting the sequence of transmit beams is interleaved in a plurality of directions into the region of interest. In an exemplary embodiment, each echo signal of the sequence of echo signals comprises a plurality of echo signals corresponding to a transmit beam in the sequence of transmit beams. In a representative embodiment, the measurement is one of a velocity measurement, a power measurement, a variance measurement, a bandwidth measurement, or a displacement measurement. In certain embodiments, the measurement is a combination of one more of a velocity measurement, a power measurement, a variance measurement, a bandwidth measurement, and a displacement measurement.

Various embodiments provide a system 100 for enhancing spatial specificity and increasing effective image acquisition speed by performing flow processing on channel data for application of nonlinear beamforming. The system 100 may comprise a plurality of transducer elements 106, 108, at least one receive beamformer 120, at least one processor 124, 128, 132, 140, and a display system 134. Each of the plurality of transducer elements 106, 108 may be operable to transmit a sequence of transmit beams in a direction into a region of interest and receive, at a plurality of depths, a sequence of echo signals corresponding to the sequence of transmit beams. The at least one receive beamformer 120 may be operable to delay clutter filtered signals to provide delay aligned clutter filtered signals. The at least one receive beamformer 120 may be operable to calculate a coherency of the delay aligned clutter filtered signals. The at least one receive beamformer 120 may be operable to nonlinearly combine the delay aligned clutter filter signals and the coherency of the delay aligned clutter filtered signals across each of the plurality of transducer elements 106, 108 at one or more depths to generate at least one beamformed signal for each received set of echo signals in the sequence of echo signals at the one or more depths. The at least one processor 128 may be configured to generate the clutter filtered signals corresponding to each echo signal of the sequence of echo signals for each of the plurality of transducer elements 106, 108 at each of the plurality of depths. The at least one processor 132, 140 may be configured to calculate a measurement for the one or more depths based on the at least one beamformed signal for each received set of echo signals in the sequence of echo signals at the one or more depths. The display system 134 may be configured to present the measurement.

In a representative embodiment, the at least one processor 124 may be configured to perform complex demodulation on each echo signal of the sequence of echo signals for each of the plurality of transducer elements 106, 108. In various embodiments, each of the plurality of transducer elements 106, 108 may be operable to transmit sequences of transmit beams in a plurality of directions into the region of interest. In certain embodiments, each of the plurality of transducer elements 106, 108 may be operable to sequentially transmit the sequence of transmit beams in a plurality of directions into the region of interest. In an exemplary embodiment, each of the plurality of transducer elements 106, 108 may be operable to interleave the transmission of the sequence of transmit beams in the direction with transmissions of sequences of transmit beams in additional directions into the region of interest. In a representative embodiment, each echo signal of the sequence of echo signals may comprise a plurality of echo signals corresponding to a transmit beam in the sequence of transmit beams. In various embodiments, the measurement may be a velocity measurement, a power measurement, a variance measurement, a bandwidth measurement and/or a displacement measurement.

Certain embodiments provide a non-transitory computer readable medium having stored thereon, a computer program having at least one code section. The at least one code section is executable by a machine for causing the machine to perform steps 200. The steps 200 may comprise generating 218 a clutter filtered signal corresponding to each echo signal of a sequence of echo signals for each of a plurality of transducer elements 106, 108 at each of a plurality of depths. The sequence of echo signals may be received by each of the plurality of transducer elements 106, 108 in response to a sequence of transmit beams transmitted by each of the plurality of transducer elements 106, 108 in a direction into a region of interest. The steps 200 may comprise delaying 219A the clutter filtered signals to provide delay aligned clutter filtered signals. The steps 200 may comprise calculating 219B a coherency of the delay aligned clutter filtered signals. The steps 200 may comprise nonlinearly combining 220 the delay aligned clutter filtered signals and the coherency of the delay aligned clutter filtered signals across each of the plurality of transducer elements 106, 108 at one or more depths to generate at least one beamformed signal for each received set of echo signals in the sequence of echo signals at the one or more depths. The steps 200 may comprise calculating 222 a measurement for the one or more depths based on the at least one beamformed signal for each received set of echo signals in the sequence of echo signals at the one or more depths. The steps 200 may comprise presenting 224 the measurement for the one or more depths at a display system 134.

In various embodiments, the steps 200 may comprise performing complex demodulation 216 on each echo signal of the sequence of echo signals for each of the plurality of transducer elements 106, 108. In certain embodiments, the sequence of transmit beams transmitted by each of the plurality of transducer elements 106, 108 may be either performed sequentially in a plurality of directions into the region of interest or interleaved in the plurality of directions into the region of interest. In an exemplary embodiment, each echo signal of the sequence of echo signals comprises a plurality of echo signals corresponding to a transmit beam in the sequence of transmit beams. In a representative embodiment, the measurement may be a velocity measurement, a power measurement, a variance measurement, and/or a bandwidth measurement.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for enhancing spatial specificity and increasing effective image acquisition speed by performing flow processing on channel data for application of nonlinear beamforming.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method comprising:
transmitting, from each of a plurality of transducer elements, a sequence of transmit beams in a direction into a region of interest;
receiving, at each of the plurality of transducer elements, a sequence of echo signals, each from a plurality of depths in the region of interest, the sequence of echo signals corresponding to the sequence of transmit beams;
generating, by at least one processor, a clutter filtered signal corresponding to each echo signal of the sequence of echo signals from each of the plurality of depths and for each of the plurality of transducer elements;
delaying, by at least one beamformer, the clutter filtered signals to provide delay aligned clutter filtered signals;
calculating, by the at least one beamformer, a coherency of the delay aligned clutter filtered signals;
nonlinearly combining, by the at least one beamformer, the delay aligned clutter filtered signals and the coherency of the delay aligned clutter filtered signals from one or more of the plurality of depths and across each of the plurality of transducer elements to generate at least one beamformed signal for each received set of echo signals in the sequence of echo signals from the one or more of the plurality of depths;
calculating, by the at least one processor, a measurement for each of the one or more of the plurality of depths based on the at least one beamformed signal for each received set of echo signals in the sequence of echo signals from the one or more of the plurality of depths; and
presenting, by the at least one processor, the measurement for each of the one or more of the plurality of depths at a display system.

2. The method of claim 1, comprising performing, by the at least one processor, complex demodulation on each echo signal of the sequence of echo signals for each of the plurality of transducer elements.

3. The method of claim 1, wherein the transmitting the sequence of transmit beams is performed in a plurality of directions into the region of interest.

4. The method of claim 1, wherein the transmitting the sequence of transmit beams is performed sequentially in a plurality of directions into the region of interest.

5. The method of claim 1, wherein the transmitting the sequence of transmit beams is interleaved in a plurality of directions into the region of interest.

6. The method of claim 1, wherein each echo signal of the sequence of echo signals comprises a plurality of echo signals corresponding to a transmit beam in the sequence of transmit beams.

7. The method of claim 1, wherein the measurement is one of a velocity measurement, a power measurement, a variance measurement, a bandwidth measurement, or a displacement measurement.

8. The method of claim 1, wherein the measurement is a combination of one more of a velocity measurement, a power measurement, a variance measurement, a bandwidth measurement, and a displacement measurement.

9. An ultrasound system comprising:
a plurality of transducer elements, wherein each of the plurality of transducer elements is operable to:
transmit a sequence of transmit beams in a direction into a region of interest, and
receive a sequence of echo signals, each from a plurality of depths in the region of interest, the sequence of echo signals corresponding to the sequence of transmit beams;
at least one receive beamformer operable to:
delay clutter filtered signals to provide delay aligned clutter filtered signals,
calculate a coherency of the delay aligned clutter filtered signals, and
nonlinearly combine the delay aligned clutter filter signals and the coherency of the delay aligned clutter filtered signals from one or more of the plurality of depths and across each of the plurality of transducer elements to generate at least one beamformed signal for each received set of echo signals in the sequence of echo signals from the one or more of the plurality of depths;
at least one processor configured to:
generate the clutter filtered signals corresponding to each echo signal of the sequence of echo signals from each of the plurality of depths and for each of the plurality of transducer elements, and
calculate a measurement for each of the one or more depths based on the at least one beamformed signal for each received set of echo signals in the sequence of echo signals from the one or more of the plurality of depths; and
a display system configured to present the measurement for each of the one or more of the plurality of depths.

10. The system of claim 9, wherein the at least one processor is configured to perform complex demodulation on each echo signal of the sequence of echo signals for each of the plurality of transducer elements.

11. The system of claim 9, wherein each of the plurality of transducer elements is operable to transmit sequences of transmit beams in a plurality of directions into the region of interest.

12. The system of claim 9, wherein each of the plurality of transducer elements is operable to sequentially transmit the sequence of transmit beams in a plurality of directions into the region of interest.

13. The system of claim 9, wherein each of the plurality of transducer elements is operable to interleave the transmission of the sequence of transmit beams in the direction with transmissions of sequences of transmit beams in additional directions into the region of interest.

14. The system of claim 9, wherein each echo signal of the sequence of echo signals comprises a plurality of echo signals corresponding to a transmit beam in the sequence of transmit beams.

15. The system of claim 9, wherein the measurement is one or more of a velocity measurement, a power measurement, a variance measurement, a bandwidth measurement, and a displacement measurement.

16. A non-transitory computer readable medium having stored thereon, a computer program having at least one code section, the at least one code section being executable by a machine for causing an ultrasound system to perform steps comprising:
generating a clutter filtered signal corresponding to each echo signal of a sequence of echo signals from a plurality of depths in a region of interest and for each of a plurality of transducer elements, wherein the sequence of echo signals is received by each of the plurality of transducer elements in response to a sequence of transmit beams transmitted by each of the plurality of transducer elements in a direction into the region of interest;
delaying the clutter filtered signals to provide delay aligned clutter filtered signals;
calculating a coherency of the delay aligned clutter filtered signals;
nonlinearly combining the delay aligned clutter filtered signals and the coherency of the delay aligned clutter filtered signals from one or more of the plurality of depths and across each of the plurality of transducer elements to generate at least one beamformed signal for each received set of echo signals in the sequence of echo signals from the one or more of the plurality of depths;
calculating a measurement for each of the one or more of the plurality of depths based on the at least one beamformed signal for each received set of echo signals in the sequence of echo signals from the one or more of the plurality of depths; and
presenting the measurement for each of the one or more of the plurality of depths at a display system.

17. The non-transitory computer readable medium of claim 16, comprising performing complex demodulation on each echo signal of the sequence of echo signals for each of the plurality of transducer elements.

18. The non-transitory computer readable medium of claim 16, wherein the sequence of transmit beams transmitted by each of the plurality of transducer elements is either:
performed sequentially in a plurality of directions into the region of interest, or
interleaved in the plurality of directions into the region of interest.

19. The non-transitory computer readable medium of claim 16, wherein each echo signal of the sequence of echo signals comprises a plurality of echo signals corresponding to a transmit beam in the sequence of transmit beams.

20. The non-transitory computer readable medium of claim 16, wherein the measurement is one or more of a velocity measurement, a power measurement, a variance measurement, a bandwidth measurement, and a displacement measurement.
